(19) United States
(12) Patent Application Publication
(10) Pub. No.: US 2007/0003996 A1
(43) Pub. Date: Jan. 4, 2007

(54) Identification of bacteria and spores

(76) Inventors: Ben A. Hitt, Wheeling, WV (US); Brian Mansfield, Catonsville, MD (US); Ping Yip, Rockville, MD (US)

Correspondence Address: COOLEY GODWARD KRONISH LLP;ATTN: PATENT GROUP, THE BOWEN BUILDING, 875 15TH STREET, N.W. SUITE 800, WASHINGTON, DC, 20005-2221, US

(21) Appl. No.: 11/350,269

(22) Filed: Feb. 9, 2006

Related U.S. Application Data
(60) Provisional application No. 60/650,979, filed on Feb. 9, 2005.
(60) Provisional application No. 60/655,470, filed on Feb. 23, 2005.

(51) Int. Cl. G06F 19/00; C12Q 1/04

(52) U.S. Cl. 435/34; 702/19

(57) ABSTRACT

Bacteria can be identified by analyzing a data stream that is obtained by processing a sample containing the bacteria, where the data stream has been abstracted to produce a sample vector that characterizes the data stream in a predetermined vector space containing at least one diagnostic cluster, the diagnostic cluster being associated with bacteria of known type, and by determining whether the sample vector rests with the diagnostic cluster, and if the sample rests within the diagnostic cluster, an indication that the bacteria are of the known type can be provided. Similarly, spores can be identified by analyzing a data stream that is obtained by processing a sample containing the spores, where the data stream has been abstracted to produce a sample vector that characterizes the data stream in a predetermined vector space containing at least one diagnostic cluster, the diagnostic cluster being associated with spores of known type, and by determining whether the sample vector rests with the diagnostic cluster, and if the sample rests within the diagnostic cluster, an indication that the spores are of the known type can be provided.

This application claims priority to U.S. Provisional Application Ser. No. 60/650,979, entitled “Low Level Detection and Differentiation of Bacillus Spores Using a Differential Mobility Spectrometer and Pattern Recognition Algorithms,” filed Feb. 9, 2005, and U.S. Provisional Application Ser. No. 60/655,470, entitled “Species-Specific Bacteria Identification Using Differential Mobility Spectrometry and Bioinformatics Pattern Recognition,” filed Feb. 23, 2005, the contents of which provisional applications are incorporated herein by reference in their entirety.

### BACKGROUND

As bacteria grow and proliferate, they release a variety of volatile compounds that can be profiled and used for speciation, providing an approach amenable to disease diagnosis through patient breath analysis. There are several chemical detectors and assays are presently being refined for use in the identification of volatile byproducts of bacterial metabolism (1, 2), which are sufficiently sensitive for analysis of volatile constituents in human breath (3, 4). Many of these breath detection technologies are focused on the measurement of volatile organic compounds, such as nitric oxide (5), ethane and pentane (6), aldehydes (7), isoprene (8), hydrogen and carbon monoxide (9), that are generated by microbes or their infected hosts in response to infection or stress.

These assays offer the potential to diagnose or follow the course of a wide variety of diseases, including chronic lung disease (10-12), and heart failure (8), with far less time, expense and invasiveness than diagnosis by microbiological typing. One experimental model of infected lung space is headspace analysis of bacteria-specific volatiles released by bacteria in liquid culture. Automated headspace concentration gas chromatography-flame ionization detection (GC-FID) analysis of several common lung pathogens reveals a number of characteristic and highly conserved dominant components (13). Gas chromatography-mass spectrometry (GC-MS) analysis of headspace volatiles has also been performed on different species of Pseudomonas bacteria, showing differences in the relative concentrations of methyl ketones, alcohols, and sulfur metabolites (14). Liquid chromatography has also been used to successfully differentiate between closely related species of Mycobacterium by the examination of various fatty acids and mycolic acid cleavage products (15). Gas chromatography has been used for the identification of Clostridium difficile, an enteric pathogen, based on different short-chain fatty acids metabolically produced by C. difficile as compared to other Clostridia (16).

A major barrier to adapting these detection methods to clinical diagnosis and other uses in the field is their technical complexity and the physical size of the analytical equipment. For this reason, a strong need exists for miniaturized, fieldable devices to analyze volatile emissions. One such device, the micromachined differential mobility spectrometer (microDMx) uses the non-linear mobility dependence of ions in high strength RF electric fields for ion filtering and detection (17, 18). Ions carried by an inert gas are passed between two planar electrodes modulated by two electric fields—an asymmetric, time dependent, periodic potential, over which a variable DC compensation voltage unique to each ion is superimposed to allow analytes to pass between the ion filter electrodes to a detector and deflector electrode (19). Similar detectors are already used daily in airports worldwide for screening hand-carried articles (20).

Previous work using microfabricated differential mobility spectrometry for bacteria classification has been coupled with pyrolysis, in which entire microorganisms are thermally degraded and either whole-cell chemistries or individual compounds specific to a species are profiled from the complex spectra produced (21,22). These works, while fundamental in studying cell compounds and identifying organisms based on their unique parts and processes, are not amenable to in vivo breath analysis applications because cell compounds released by pyrolysis would not be released as volatiles under normal physiological conditions.

In addition, the traditional approach of peak identification, which works well for quantitatively and qualitatively analyzing MS and FID generated data, becomes problematic with differential mobility detection. When mixtures are analyzed with ion mobility spectrometers preferential ionization of one or more of the components may interfere with the formation of product ions of other components in the sample. For example, when four or more ionized ingredients are mixed together it results in the loss of some individual peaks and/or the coalescence of individual peaks (23). This behavior may explain why a correlation between molecular structure and compensation voltage in these types of devices has not yet been determined (19) despite the thorough theoretical and experimental modeling of resolution and sensitivity (24).

Identification of organisms based on a set of consistent compounds is also flawed, in that production of volatile compounds is dependent on the dynamics of the whole ecosystem (21). Individual species generate a reproducible profile for volatiles only within consistent environmental parameters. Changes in growth conditions can change the volatile profile for a given species. Moreover, the addition of other organisms can complicate the profile as volatiles released by these “contaminants” can act as a mode of communication, inducing changes in the target organisms volatile compound production (25), changing the expected volatile profile of the target organism.

With increasing concern about the potential for a biological agent attack, the need for a portable, inexpensive, and durable sensor that can rapidly detect and identify biological weapons agents continues to grow. B. anthracis, the causative agent of anthrax, has been identified as one of the most dangerous disease-causing organisms capable of devastation in the event of a release (52). Anthrax spores can be inhaled and transported to lymph nodes, germinating up to 60 days later (53). The germinating bacteria produce a toxin that causes necrosis, edema, and hemorrhaging (54, 55). In the event of a release, the rapid detection of the presence of anthrax is critical for effectively treating patients that have been exposed (56). Quickly identifying the presence of environmental spores is difficult for several reasons: the DNA is well-protected inside the spore, various serotypes exist, and the spore structure is biochemically different from that of vegetative cells (57-60). Furthermore, B. anthracis is genetically similar to other Bacillus species, such as B. cereus and B. thuringiensis (61, 62), complicating the differentiation of the potential biological weapon from non-pathogenic spores.

Since the October 2001 anthrax attacks in the United States, there has been significant research focused on finding a sensitive and specific anthrax detection system. To date, most work has focused on nucleic acid detection (63-75), which offers extremely high sensitivity. However, the spores must be at least partially germinated prior to the assay, several reagents are required, and assay times are still around a half-hour or more for a sample with few spores. Another detection method that has been widely explored is immunodetection (57-59, 64, 76-80). Again, these assays can be very sensitive but require various reagents and still typically take 30 minutes or longer. Another concern is the cross-reactivity of the antibodies used. Mass spectrometry has also been used to detect spores (81-86), but the sensitivity is not as high as with nucleic acid or antibody detection. In addition, mass spectrometers continue to remain very expensive, limiting their potential for field-use.

To date, few detection levels below 10⁵ spores have been reported. The ID 50 (median infectious dose) has been reported to be 8,000 to 10,000 spores (87). The LD 50 (median lethal dose) has been reported at 61,800 spores in Rhesus macaques (88). Arakawa, et al. reports detection of 1,000 spores using microcalorimetric spectroscopy (89), but this technique fails in the presence of water and thus requires sample lyophilization prior to analysis.

There is an urgent need for a small, inexpensive, robust sensor that can rapidly detect bacteria and other microorganisms that release volatile compounds as well as for the detection of bio-warfare agents. For example, in the event of an intentional release, Bacillus anthracis, the causative agent of anthrax, would be one of the most perilous disease-causing organisms. Currently, much of the anthrax detection research is concentrated on nucleic acid detection, immunoassays and mass spectrometry, with few detection levels reported below 10⁵ spores.

### SUMMARY

Bacteria can be identified by analyzing a data stream that is obtained by processing a sample containing the bacteria, where the data stream has been abstracted to produce a sample vector that characterizes the data stream in a predetermined vector space containing at least one diagnostic cluster, the diagnostic cluster being associated with bacteria of known type, and by determining whether the sample vector rests with the diagnostic cluster, and if the sample rests within the diagnostic cluster, an indication that the bacteria are of the known type can be provided. Similarly, spores can be identified by analyzing a data stream that is obtained by processing a sample containing the spores, where the data stream has been abstracted to produce a sample vector that characterizes the data stream in a predetermined vector space containing at least one diagnostic cluster, the diagnostic cluster being associated with spores of known type, and by determining whether the sample vector rests with the diagnostic cluster, and if the sample rests within the diagnostic cluster, an indication that the spores are of the known type can be provided.

### BRIEF DESCRIPTION OF THE DRAWINGS

FIG. 1 shows the response of the positive ion channel of the detector in GC-microDMx set up for bacteria headspace analysis using ketone test standards.

FIG. 2 illustrates growth curves for the species analyzed.

FIG. 3 shows representative spectra for M smegmatis at various stages its growth cycle.

FIG. 4 shows representative GC-MS Total Ion Chromatographs for E. coli incubated for different periods of time.

FIG. 5A shows averaged aligned spectra for M smegmatis (MS), B. thuringiensis (BT), B. subtilis(BS), and E. coli(EC).

FIG. 5B shows the spectra of FIG. 5A with averages subtracted from each other and biomarkers overlaid.

FIGS. 6A-6C show the distribution of features across 40 models for B. subtilis versus B. thuringiensis; B. subtilis versus B. cereus; and B. cereus versus B. thuringiensis, respectively

FIG. 7 is a plot of the dominant classifier feature 18097.

FIGS. 8A-8C show representative microDMx spectra for B. subtilis; B. cereus; and B. thuringiensis, respectively.

### DETAILED DESCRIPTION

As bacteria grow and proliferate, they release a variety of volatile compounds that can be profiled and used for speciation, providing an approach amenable to disease diagnosis through patient breath analysis. As a practical alternative to mass spectroscopy detection and whole cell pyrolysis approaches, the present invention relates to a methodology that, in one aspect, involves detection of such volatile compounds via a sensitive, micromachined differential mobility spectrometer (microDMx™) that is capable of operating at ambient temperature and at atmospheric pressure.

Recently, sophisticated bioinformatics algorithms (Correlogic Systems, Inc.®) have been applied to serum proteomic patterns for detection of prostate (26, 27) and ovarian cancer (28, 29) biomarkers. This technology is described in U.S. Pat. No. 6,925,389 and Published U.S. Application 2002/0046198 (the disclosures of which are hereby incorporated by reference).

The disclosed methodology analyzes bacteria headspace using (1) a small, sensitive, and inexpensive detector, and (2) sophisticated data analysis that will allow classification of bacterial species despite sample-to-sample variability within a species set. Bacteria selected for these experiments included Escherichia coli, Bacillus subtilis, Bacillus thuringiensis, an agent in opportunistic respiratory infections, and Mycobacterium smegmatis, a surrogate for Mycobacterium tuberculosis.

Pattern discovery/recognition algorithms (ProteomeQuest®) are applied to analyze headspace gas spectra generated by microDMx to reliably discern multiple species of bacteria in vitro, for example, Escherichia coli, Bacillus subtilis, Bacillus thuringiensis and Mycobacterium smegmatis. The overall accuracy for identifying volatile profiles of a species within the 95% confidence interval for the two highest accuracy models evolved was between 70.4% and 89.3% based upon the coordinated expression of between 5 and 11 features. Identification of organisms based on a set of consistent compounds is also flawed, in that production of volatile compounds is dependent on the dynamics of the whole ecosystem (21). Individual species generate a reproducible profile for volatiles only within consistent environmental parameters. Changes in growth conditions can change the volatile profile for a given species. Moreover, the addition of other organisms can complicate the profile as volatiles released by these “contaminants” can act as a mode of communication, inducing changes in the target organisms volatile compound production (25), changing the expected volatile profile of the target organism. This makes the identification of an infection in breath samples based on a set of consistent compounds inefficient.

The approach disclosed below produces variability in volatiles released within each species set, and data analysis that allows a person skilled in the art to ignore this variability and find markers that distinguish between species only. Such data analysis algorithm will efficiently cycle through various features in the volatile profiles and pick out those features that are constant within a set and that best distinguish sets of data from each other.

Bacillus spores can be detected in water to a level below the reported ID 50 and closely-related species can be differentiated using microDMx™ and ProteomeQuest®. The sensitivity of this device combined with the powerful algorithms identified above surprisingly allows exceptional real-time detection of bacterial spores in addition to bacteria. As disclosed below, a person skilled in the art can detect Bacillus spores down to a level below the reported median infectious dose (ID 50) of B. anthracis and can distinguish between closely-related species. For example, markers were be identified that distinguish three species of Bacillus after injections of 5,000 to 80,000 organisms.

A. Analysis of Bacteria

Reagents. 2-butanone, 2-pentanone, 2-heptanone, 3-octanone, 3-nonanone, 2-decanone were purchased from Sigma Aldrich (St. Louis, Mo.) and used as received. Bacterial strains (E. coli DH5α ATCC 53868, B. subtilis ATCC 23857, B. thuringiensis ATCC 10792 and M. smegmatis ATCC 700084 and 700738) were obtained from American Type Culture Collection (Manassas, Va.). Lowenstein-Jensen medium slants were purchased from Becton, Dickinson and Company (Franklin Lakes, N.J.). Luria-Bretani (LB) was obtained from Difco Laboratories (Franklin Lakes, N.J.). Agar was obtained from EM Science (Gibbtown, N.J.).

GC-microDMx Instrumentation. The experimental setup consisted of an Agilent

Headspace Sampler (Agilent Technologies, Palo Alto, Calif.) connected to the inlet of an HP 5890 II GC (Agilent Technologies). The GC was equipped with a 10 m HP VOC fused silica column with 0.32 mm ID, and 1.8 μm biphenyl methyl siloxane film (Agilent Technologies) to allow a nominal pre-separation of analytes. A differential mobility spectrometer (microDMx) (Sionex Corporation, Waltham, Mass.) was connected to the detector outlet of the GC. Grade 5 Nitrogen was used as the carrier gas to sweep the headspace sample from the culture vials in the headspace sampler through a transfer line into a silica column and carry it into the microDMx. The sample carrier flow was regulated by the headspace sampler and it joined a second flow of Nitrogen at 300 ml/min regulated by a mass flow controller (MKS Instruments, Andover, Mass.), for introduction into the microDMx. The headspace sampler oven was set to 60° C., the sample loop to 75° C., and the transfer line to 85° C. The GC inlet was set to 100° C., the GC oven operated on a ramp program starting with a 3 minute hold at 60° C., a ramp of 6°/min to 140° C., and a 2 minute hold at 140° C. The GC detector heating block was set to 140° C. Sample vials were heated in the GC oven for 15 minutes at 60° C. with slow agitation to release compounds into the headspace. The vials were pressurized for 0.10 minutes at 15.2 psi, loop fill time was 0.5 minutes, loop equilibration time was 0.05 minutes, and the injection time was 0.5 minutes. The microDMx compensation voltage swept through a voltage range from −35 to 5 Volts every 0.65 seconds. The RF field was set at 1,200 Volts. Spectra corresponding to detected positive and negative ions are recorded on a laptop computer connected to the microDMx unit.

Standards. The detector sensitivity within this setup was tested using ketone standards (n=5 each). A dilution series of 1 ppm mixture of 2-butanone, 2-pentanone, 2-heptanone, 3-octanone, 3-nonanone, and 2-decanone was prepared in deionized water. The standards were also tested in a 5973 Mass Spectrometer (Agilent Technologies) with a Gerstel Multipurpose Sampler (Gerstel Inc., Mülheim, Germany) and the same Helium carrier gas flow, time, and temperature parameters. For each concentration tested, the six ketone peaks on GC-microDMx spectra were located by their absolute maxima points. Intensity was recorded for the compensation voltage of the peak maxima, which occurred between +2 V and −7 V, as well as for a background measurement at compensation voltage −34V for the retention time of the peak maxima. Background measurements were subtracted from their corresponding peak maxima, baseline subtracted intensities were averaged over five runs, and standard errors were calculated for each ketone at each concentration.

Bacteria Preparation. E. coli DH5α, B. subtilis and B. thuringiensis were grown overnight at 37° C. on Luria-Bretani (LB) agar and single colonies were used to inoculate 20 ml of LB broth. The liquid cultures were incubated at 37° C. with 180 rpm shaking for 18 hours. Then 100 μl of these batch cultures were used to inoculate 10 ml of LB in 20 ml headspace vials (Agilent Technologies). Headspace vials were capped with autoclaved septa and aluminum caps and returned to the incubator for 1-9 hours. Two strains of M. smegmatis were plated on Lowenstein-Jensen Medium Slants and incubated at 37° C. for 42 hours. 20 ml of LB broth were inoculated with single colonies and incubated at 37° C. with shaking for 42 hours. Headspace vials were then inoculated as above and incubated 1-32 hours. Over 100 headspace samples for each bacteria species were autosampled by GC-mircoDMx.

Bacteria Culture Characterization. The optical densities of the cultures were measured in a Cary 300 Bio UV-Visible Spectrophotometer (Varian, Palo Alto, Calif.) at 600 nm at 40 minute intervals in 1 ml disposable optical polystyrene cuvettes (VWR International, West Chester, Pa.). Duplicate samples were tested for each species. E. coli cell densities were approximated by plating dilutions of a culture grown for five hours in a headspace vial.

The headspace of E. coli, incubated over different periods in septum capped vials as described for GC-MicroDMx experiments, were further characterized using mass spectroscopy and Solid Phase Microextraction (SPME). Extraction of the volatile organic compounds in the headspace was performed using a 65 μm Polydimethylsiloxane/Divinylbenzene (PDMS/DVB) coating of a SPME Fiber Assembly (Supelco, Bellefonte, Pa.) for one hour at 60° C. The GC conditions were as follows: desorption for 5 minutes at 250° C.; oven at 50° C. for 5 minutes, ramp of 25°/min to 100° C. with a hold for 4 minutes, 10°/min to 150° C. for 6 minutes, 5°/min to 205° C. up to 40 minutes. An HP-5MS 30 m fused silica column with 0.25 mm ID and 0.25 μm film was used (Agilent Technologies). The injection was in splitless/split mode, closed for 5 minutes at 250° C., with a SPME inlet liner.

Data Analysis. The three-dimensional data sets that include compensation voltage (V_{c}), GC retention time and signal intensity were plotted and processed using MATLAB 6.5.1 release 13. (Mathworks, Natick, Mass.). Spectra were aligned in the compensation voltage dimension because V_{c} can be affected by moisture content and slight gas flow rate fluctuations (17, 31). From each run, positive and negative spectra were concatenated. They were then aligned in the V_{c}—dimension by a rigid shift of a few pixels or less as necessary, as determined by a maximum cross-correlation value. A single reference file was used for all files for this alignment. Then, all files were interpolated to contain the same number of scan lines.

Analysis that combines genetic algorithm elements first described by Holland (32) with cluster analysis elements described by Kohonen (33) was used to examine the microDMx spectra. Between 108 and 124 spectra for each species were randomly distributed into groups of 25 files for training, 50 files for testing, and the remainder for independent validation of the models. Models were generated using the ProteomeQuest® (Correlogic Systems, Inc., Bethesda, Md.) software package, which utilizes a combination of lead cluster mapping and a genetic algorithm to rapidly identify informative combinations of features (which form the models) in complex data sets as described previously (26-30, 34). A number of models were built in which adjustable parameters were scanned across a range of values to find the best combination. The number of features in each model was varied between 5 and 12. The Match parameter, which is a measure of the size of the decision boundary around each cluster, was scanned across the range 0.5 (large boundary) to 0.9 (small boundary). The Learn Parameter was set to 0.2. and the Population, representing the number of combinations of features assessed for each model, was set to 20,000. Each model cycled through the genetic algorithm until there was no improvement in the model accuracy for 50 consecutive iterations.

### RESULTS AND DISCUSSION

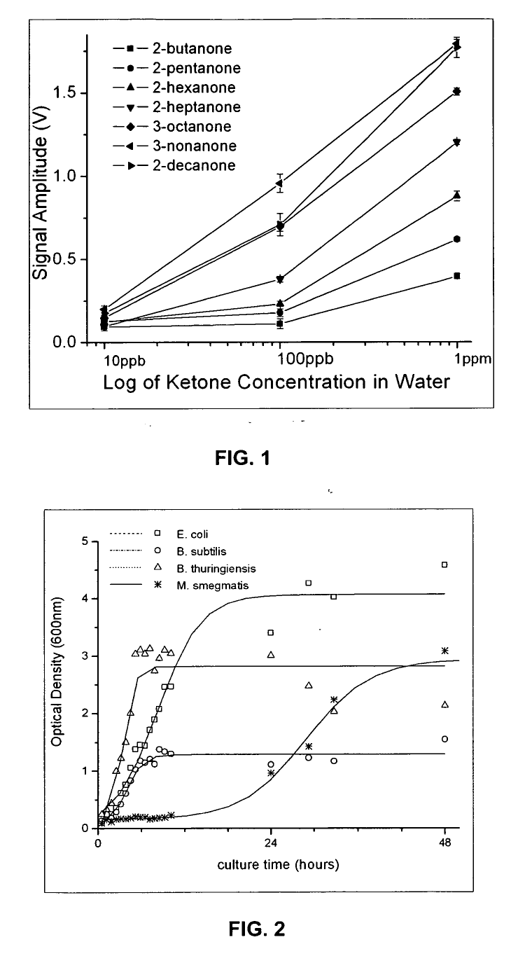

GC-microDMx sensitivity. The sensitivity of the setup was determined by analyzing spectra for ketone standards at 1 ppm to 1 ppb concentrations in liquid. Maximum peak intensities for each ketone at each concentration were found and a value for estimated file background was subtracted. All positive ion spectra contain two carrier gas (nitrogen) peak lines around −16 V and −22 V. The response curves of the positive ion channel of the microDMx detector are shown in FIG. 1. The reproducibility was consistent over a two week period and standard error was less than 3.5% for 1 ppm, and less than 28% for 100 ppb and 10 ppb. The signal could not be distinguished above background at ketone concentrations under 10 ppb. The sensitivity of our setup was comparable to mass spectrometry detection, under the same conditions. The GC-MS detected down to 100 ppb ketone concentrations, by sampling the headspace using the same GC parameters as the GC-microDMx. High sensitivity of our setup to ketones is advantageous because these chemicals are often included in libraries of bacteria volatiles (13, 14, 25 33-37) as well as in exhaled breath of patients with various disorders including diabetes (38), epilepsy (39), liver dysfunction and lung cancer (40).

Bacteria Characterization. The disclosed method created variability in volatile profiles within each species set to ensure that the bioinformatics approach is capable of finding biomarkers that were consistent in every file despite this variability. Growth curves for the organisms, shown in FIG. 2, indicate that under these culture conditions, B. thuringiensis was in lag phase for approximately one hour and in exponential growth for 5.2 hours before entering stationary phase. Similar results were found for B. subtilis which was in lag phase for an hour, and in exponential phase for about 5.8 hours. E. coli cultures remained in lag phase for one hour, but exponential growth continued up to 9.3 hours. Lag phase for M. smegmatis was 9 hours, with stationary phase reached only after 33 hours of growth. During the exponential phase, the doubling times were 5.8 hours for M. smegmatis, 1.8 hours for B. thuringiensis, 1.9 hours for B. subtilis, and 2.5 hours for E. coli. These doubling times are longer than expected, likely because they were growing in an environment with minimal oxygen transfer. At the midpoint of the exponential phase the optical density for E. coli was 1.55 absorbance units, which translated to 3×10⁸ colony forming units (CFU) per ml, on the order of M. tuberculosis bacteria found in a tuberculosis cavity, 10⁷ to 10⁹ organisms (41).

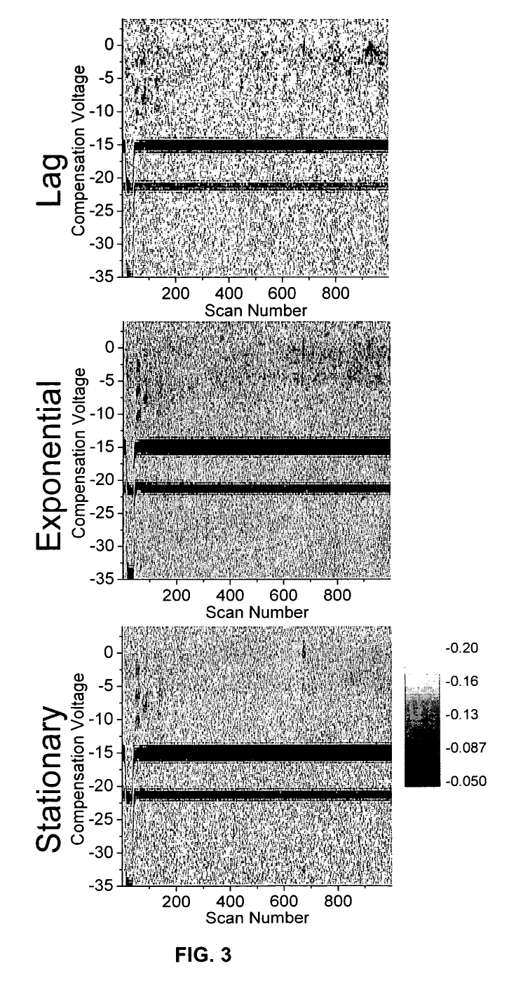

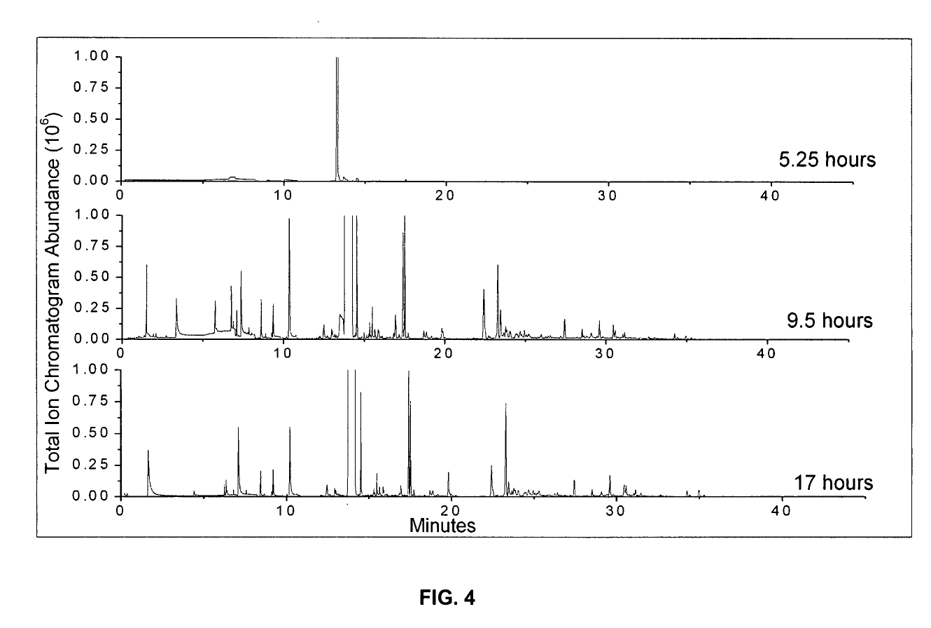

FIG. 3 shows representative microDMx spectra for M. smegmatis during the various phases of the growth curves. The profiles generated from cells cultured for different periods of time appear slightly different: many peaks begin to appear after the lag phase for all species, new peaks appear in B. subtilis, B. thuringiensis, and M. smegmatis in the stationary phase, while some peaks from the exponential plots are not visible in the stationary spectra. Besides these noticeable differences, there may be profile variations due differences in relative concentrations of the volatiles and due to volatiles of low enough concentrations that they are not easily visible. These differences are highlighted for E. coli cultured for different periods by the GC-MS profiles in FIG. 4, where new peaks appear, other peaks disappear, and relative ratios of peaks visibly change with time. The data are consistent with the idea that at different parts of a growth curve, different numbers of cells are moving through cell cycles at various rates and a number of cells are dying, both of which involve different pathways (42) and potentially release different metabolic volatiles. Other effects that play a role in profile changes within a single data set include volatile interactions that are not fully understood, as well as day to day environmental changes that can impact microDMx detection.

These factors are relevant for breath analysis applications. Breath exhalate is composed of many volatiles that interact with each other and create unique fingerprints. Variations in each person's natural flora, environmental chemical exposure, and various infections that may be taking place at the same time determine the ecosystem of a target microorganism and may become part of the interfering volatile signal.

Bacteria volatiles pattern recognition. Over 100 headspace gas measurements were made for E. coli, B. subtilis, B. thuringiensis, and M. smegmatis. Spectra from the microDMx were generated for each bacteria species and randomly divided into a training set, a testing set, and a validation set. Using the training samples as a reservoir for features and testing samples for assessing the features, multiple four-way comparison models were evolved that were validated with the remaining independent samples. The quality of the models was judged primarily on the accuracy of correctly classifying a validation sample into one of the four species. The highest overall accuracy model (A) was 84.2% accurate in identification of all validation set spectra. Another model with high accuracy and a low number of nodes (B) was 77.8% accurate. Details of A and B are summarized in Table 1 and the two models are compared in Table 2. The 95% confidence intervals calculated for validation accuracy of each species are based on the efficient-score method described by Newcombe (43). The overall accuracy within the 95% confidence interval for both models was between 70.4% and 89.3%.

TABLE 1
Validation of top accuracy models built
for identifying volatiles profiles

| | | samples tested | | | |
|---|---|---|---|---|---|
| | | | B. thurin- | | M. smeg- |
| | | B. subtilis | giensis | E. coli | matis |
| model A | | | | | |
| samples | B. subtilis | 35 | 3 | 1 | 0 |
| identified | B. thurin- | 4 | 36 | 2 | 3 |
| | giensis | | | | |
| | E. coli | 0 | 1 | 32 | 0 |
| | M. smeg- | 10 | 0 | 1 | 30 |
| | matis | | | | |
| total validation | | 49 | 40 | 36 | 33 |
| samples | | | | | |
| validation | | 71.4 | 90.0 | 88.9 | 90.9 |
| accuracy (%) | | | | | |
| 95% confidence | | 56.5-83.0 | 75.4-96.7 | 73.0-96.4 | 74.5-97.6 |
| interval (%) | | | | | |
| model B | | | | | |
| samples | B. subtilis | 26 | 2 | 1 | 1 |
| identified | B. thurin- | 16 | 34 | 0 | 2 |
| | giensis | | | | |
| | E. coli | 1 | 0 | 33 | 0 |
| | M. smeg- | 6 | 4 | 2 | 30 |
| | matis | | | | |

| | | | | |
|---|---|---|---|---|
| total validation | 49 | 40 | 36 | 33 |
| samples | | | | |
| validation | 53.1 | 85.0 | 91.7 | 90.9 |
| accuracy (%) | | | | |
| 95% confidence | 38.4-67.2 | 69.5-93.8 | 76.4-97.8 | 74.5-97.6 |
| interval (%) | | | | |

TABLE 2
Comparison of two top models validated

| | overall | 95% confidence | | | |
|---|---|---|---|---|---|
| model | accuracy | interval | features | nodes | match |
| A | 84.2% | 77.3-89.3% | 11 | 56 | 0.9 |
| B | 77.8% | 70.4-83.9% | 5 | 7 | 0.8 |

While model A was based on II features with a tight decision boundary (match=0.9) around each of the 56 nodes in the cluster map, model B was composed of 5 features, 7 nodes, and a slightly larger decision boundary match of 0.8. Different models provide some choices: here, the model with the highest accuracy has more nodes with more stringent decision boundaries, while another model with slightly lower accuracy has fewer nodes and but less tightly clustered data. Theoretically, a more robust model would have fewer nodes, which means that more samples from the same group fall into the same nodes, although high node models have been observed to be robust over time across many samples. The optimal characteristics for long term validity of models can not be defined until the models are tested over time, as the true test of any model is how well it continues to work when challenged with more new data.

In developing a methodology for classifying bacterial volatiles, very diverse bacteria (Mycobacteria, acid-fast rods with generation time on the scale of hours, versus Bacillus species which are endospore forming, Gram-positive rods with generation time on the scale of minutes) that could inhabit the pulmonary environment were selected. Two organisms of the same genus were also studied to see how well closely related species could be distinguished. The bioinformatics approach to classification worked consistently for all species in categorizing samples of both similar and different bacteria species.

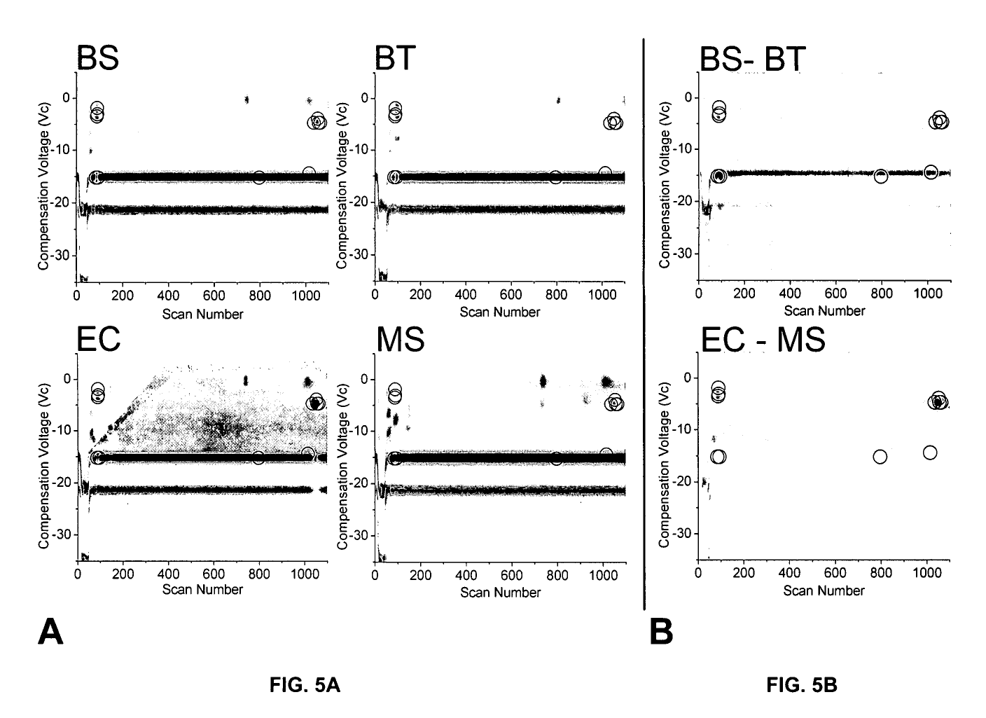

The locations of the 11 biomarker features of model A are overlaid on averaged spectra in FIG. 5A. Features of model B (not shown) are spread over the spectrum in a similar fashion. The features selected for classification do not appear easily distinguishable as specific peaks. By cycling through thousands of randomly selected locations in the spectra and by making richer classification decisions based on ranges of intensities found at these locations, the bioinformatics approach allows an efficient search for features that are of very low intensity (low volatile concentrations) and for features that represent compound that are detected only at consistent compensation voltages despite changes in concentration or changes in headspace component molecules. This approach also allows sample-to-sample profile differences to be ignored, focusing on species-to-species differences. For example, E. coli are known to release the compound indole as a metabolic byproduct (44, 45).

One route toward classification is to attempt to identify the location of the indole peak on the microDMx spectra and test for this organism using the peak. Headspace of pure indole was tested in the disclosed setup, and it was found that indole elutes through the column at approximately 1045 scans and at a compensation voltage of −4.6. However, the peak in cultured E. coli which is believed to correspond to indole appeared at similar but not identical locations of exponential and stationary phases, and did not appear at all in the lag phase of batch cultures. Since this chemical has a very high abundance in exponential and stationary phases, and the peak for it is strongest relative to all other peaks, the peak can be tracked without sophisticated analysis. But when spectra of organisms like B. subtilis and B. thuringiensis in FIG. 3 are examined, no unique robust peaks are immediately obvious. Low intensity peaks for volatiles of these organisms may be convoluted in the background noise. If peaks for specific volatiles align perfectly with each other in different mixtures, then a simple analysis may consist of averaging spectra of each species and subtracting the averages to find differences in their volatile signatures. In the present case, different volatiles present in headspaces of bacteria cultured for different periods of time may interact with each other in different ways to produce slight shifts of peaks, resulting in failure of such analysis.

In FIG. 5B, when B. thuringiensis averaged spectra are subtracted from B. subtilis averaged spectra, barely any visible differences between the species appear. Without the pattern recognition algorithm, this data could not be resolved into two different species. The disclosed approach allows the variability that can be attributed to presence of volatiles released from additional microbes in a bacteria mixture studies, or variation in breath chemistries in clinical studies, to be disregarded.

This type of volatiles sampling and data processing should be applicable in engineering and medicine as a pulmonary disease diagnostic tool. The GC-microDMx system could be manufactured as a portable device with the hand held microDMx detector and a silicon chip based microfabricated GC column (46) as high speed capillary columns have already been coupled to ion mobility spectrometers to achieve pre-separation of mixtures of breath volatiles (47). This data analysis can identify biomarkers from sample sets that have complicated signals by focusing only on differences between an infected and a control group while disregarding differences within a group. Precise identification of individual compounds released by microorganisms is not a viable option in clinical applications, in which identification of these compounds will be confounded by other chemicals to which patients have been exposed, as well as the interaction of these compounds and volatiles from other bacteria that shift spectra, preventing simple peak identification.

The disclosed GC-microDMx method allows sampling headspace of bacteria cultures to generate volatile profiles for different species. The highly sensitive, potentially portable microDMx detection, is preferably coupled with sophisticated data analysis. Bioinformatics pattern recognition process has been successfully applied to find markers that identify bacterial species based on their volatile signatures from different phases of their growth curves. This type of data analysis allows inclusion of variables into a set, which can be expanded from one species in different growth phases, to one species in different culture environments, to multiple species in one culture, and so on. With instrumentation that can easily be made into a field employable device and data analysis techniques that take into account variability within a sample set, this methodology can be applied to evaluating breath samples of a diseased and healthy population to find markers to distinguish the two. Other applications may include detection and identification of microbial growth in building materials (48-50) and veterinary uses (51).

B. Analysis of Bacterial Spores

Spore Preparation. B. subtilis strain SMY, a wild-type, prototrophic, Marburg strain (obtained from P. Schaeffer) (90), was pre-grown overnight at 30° C. on a plate of tryptose blood agar base (Difco Laboratories; Franklin Lakes, N.J.) and used to inoculate 2-L of DS medium (91) in a 6-L Erlenmeyer flask. The flask was incubated with shaking (200 rpm) at 37° C. for 48 hours. The cells were harvested by centrifugation at 13,000×g for 20 minutes at 4° C., washed four times with 100-ml sterile, deionized water, and resuspended in 20-ml sterile water. The suspension was estimated to contain 95% mature, refractile spores by phase contrast microscopy. The spore titer was determined by assaying colony formation on DS agar plates after heating to 80° C. for 10 minutes. Spores were diluted in sterile water when lower concentrations were required for testing. B. cereus strain CIP5832 and B. thuringiensis strain 407 Cry+ (both obtained from D. Lereclus, Institut Pasteur, Paris, France) were grown on DS agar plates for 48 hrs at 37° C. The cultures were harvested by flooding the plates with sterile, deionized water and scraping up the bacterial colonies. After transfer to a centrifuge tube and centrifugation at 13,000× g for 10 min at 4° C., the spores were washed, resuspended, and titered as above.

Pyrolysis-FAIMS (High-Field Asymmetric Waveform Ion Mobility Spectrometry) Analysis of Bacillus Spores. The experimental setup consisted of a CDS Pyroprobe 1000 (CDS Analytical, Inc., Oxford, Pa.) connected to the inlet of an HP 5890 Gas Chromatograph (GC) (Agilent Technologies, Palo Alto, Calif.). The GC was equipped with a 0.5 m deactivated fused silica column (Agilent). A prototype SDP-1 micromachined differential mobility spectrometer (microDMx) (Sionex Corporation, Waltham, Mass.) was connected to the detector outlet of the GC. Grade 5 Nitrogen was used as the carrier gas to sweep the pyrolyzed sample from the pyrolysis chamber into the deactivated fused silica column and carry it into the microDMx. The flow was regulated by mass flow controllers (MKS Instruments, Andover, Mass.), and was set to 30 ml/min for the sample to be carried through the pyrolyzer and GC column, where it joined a second flow of nitrogen at 300 ml/min for introduction into the microDMx. The interface temperature of the pyrolyzer was set at 110° C., the GC inlet was set to 150° C., the GC oven was held constant at 200° C., and the GC detector heating block was set to 150° C.

A slurry of 4 μl of Bacillus spores suspended in sterile water was loaded into a quartz tube. The tube was placed in the pyrolysis probe platinum coil, and the probe was then loaded into the pyrolysis unit. The spores were then pyrolyzed by ramping the temperature up to 650° C. at a rate of 0.01° C./msec, and then holding at this temperature for 99.99 seconds. The microDMx was programmed to have the compensation voltage sweep through a voltage range from −40 to 10 Volts every 1.6125 seconds. The RF field was set at 1200 Volts. The spectra of the pyrolyzed spores corresponding to the detected positive and negative ions were recorded on a laptop computer connected to the microDMx unit.

For each of the three species, B. subtilis, B. cereus, and B. thuringiensis, 100 experiments for each of three concentrations (900 experiments total) were conducted as described. The concentrations used were 2e+7 spores/ml (80,000 spores/experiment), 2.5e+6 spores/ml (10,000 spores/experiment), and 1.25e+6 spores/ml (5,000 spores/experiment). The positive and negative spectra from each run were concatenated and then aligned across all runs so that the pyrolysis event starting point occurred at exactly the same scan in each file. Additionally, the data was aligned in the Vc-dimension by a rigid shift of a few pixels or less when necessary, as the compensation voltage at which an ion elutes can be affected by the moisture content of the sample and the gas flow rate as it passes through the microDMx (92, 93). The amount of shift was determined by comparison of the total abundances at each Vc value (across all scans) of a data file with these total abundances from a single reference file. The cross-correlation of the data and reference files was calculated to determine optimal alignment, based on the location at which this value was at a maximum. The positive and negative data are then rigidly shifted in the Vc direction based on this result. The data were then analyzed by ProteomeQuest® (Correlogic Systems Inc.), a proprietary pattern recognition software package that combines genetic algorithm elements first described by Holland (99) with cluster analysis elements described by Kohonen (100), as previously described (94-98).

### Results and Discussion

A total of n=100 pyrolysis-microDMx experiments were conducted for each B. subtilis, B. cereus, and B. thuringiensis spore species at three concentrations, after method development to determine the optimal conditions for biomarker release (101). The data from each species was randomly divided into three categories: a training set (50 spectra of each species), a testing set (150 spectra of each species), and a validation set (˜100 spectra of each species). The training and testing sets consisted of files whose species identities were known by the computer. Lead cluster maps generated using the training set were tested for accuracy by the testing set. Following the ranking of the lead cluster maps, genetic recombination between map markers shuffled the most informative markers. The process of lead cluster mapping and recombination was then iterated until 50 consecutive cycles showed no further improvement in accuracy. The validation set, which was withheld from the modeling process, was then scored by the model to give an independent measure of the accuracy of the model on blinded data. The specificity, sensitivity, and accuracy described below were calculated from the results of the independent validation set using the following equations:
Sensitivity=(True Positives)/(True Positives+False Negatives)
Specificity=(True Negatives)/(True Negatives+False Positives)
Accuracy=(True Positives+True Negatives)/(Total Number of Samples)

The files were first compared in binary groups consisting of a single species at all three concentrations compared to a second single species at all three concentrations, and models were created that allowed the differentiation of one species from another. The results from six models giving the highest accuracies are shown in Table 3, which shows comparisons of validation results based on two-way modeling across all concentrations (80 k spores, 10 k, and 5 k). 101 B. cereus, 99 B. subtilis, and 100 B. thuringiensis files were used. Data are shown for each binary comparison include number of biomarkers (B), match (M), number of nodes (N), sensitivity (Sn), specificity (Sp), and percent accuracy (A). Sensitivity and specificity are calcilated with respect to the first species named in each comparison. B. subtilis was readily distinguished from B. cereus and also from B. thuringiensis even at a level as low as 5,000 spores, with accuracies higher than 90%. B. cereus and B. thuringiensis proved slightly more difficult to distinguish, with accuracies just under 70%. However, this is not surprising, as these two species are genetically very similar. The specificities and sensitivities for each model are also reported in this table. For example, for the model with the highest accuracy (92.0%) in the comparison of B. cereus and B. subtilis, the sensitivity and specificity for the files of each species used in validation were 87.9% and 96%, respectively, as calculated with respect to B. cereus. This means that for the 101 B. cereus files in the blind testing, 89 were classified as B. cereus and the remaining 12 were classified as B. subtilis, whereas of the 99 B. subtilis files, 95 were classified correctly while 4 were classified as B. cereus.

TABLE 3
Comparisons of validation results based on two-way modeling

| | | | | | | |
|---|---|---|---|---|---|---|
| Model | B | M | N | Sn | Sp | A |

B. subtilis & B. thuringiensis

| | | | | | | |
|---|---|---|---|---|---|---|
| 1 | 8 | 0.9 | 21 | 99.0 | 98.0 | 98.5 |
| 2 | 12 | 0.9 | 24 | 93.0 | 99.0 | 96.0 |
| 3 | 9 | 0.9 | 24 | 93.0 | 96.0 | 94.5 |
| 4 | 10 | 0.9 | 16 | 91.0 | 96.0 | 93.5 |
| 5 | 9 | 0.8 | 4 | 89.0 | 96.0 | 92.5 |
| 6 | 6 | 0.9 | 16 | 88.0 | 96.0 | 92.0 |

B. cereus & B. subtilis

| | | | | | | |
|---|---|---|---|---|---|---|
| 1 | 9 | 0.9 | 33 | 87.9 | 96.0 | 92.0 |
| 2 | 2 | 0.9 | 36 | 91.9 | 91.1 | 91.5 |
| 3 | 11 | 0.9 | 35 | 90.9 | 91.1 | 91.0 |
| 4 | 10 | 0.9 | 29 | 90.9 | 90.1 | 90.5 |
| 5 | 7 | 0.9 | 13 | 94.9 | 86.1 | 90.5 |
| 6 | 8 | 0.8 | 7 | 96.0 | 82.2 | 89.0 |

B. cereus & B. thuringiensis

| | | | | | | |
|---|---|---|---|---|---|---|
| 1 | 6 | 0.8 | 5 | 76.0 | 62.4 | 69.17 |
| 2 | 8 | 0.7 | 3 | 72.0 | 66.3 | 69.14 |
| 3 | 12 | 0.7 | 2 | 81.0 | 55.4 | 68.14 |
| 4 | 10 | 0.8 | 10 | 64.0 | 71.3 | 67.67 |
| 5 | 9 | 0.7 | 2 | 70.0 | 63.4 | 66.68 |
| 6 | 7 | 0.7 | 3 | 61.0 | 69.3 | 65.17 |

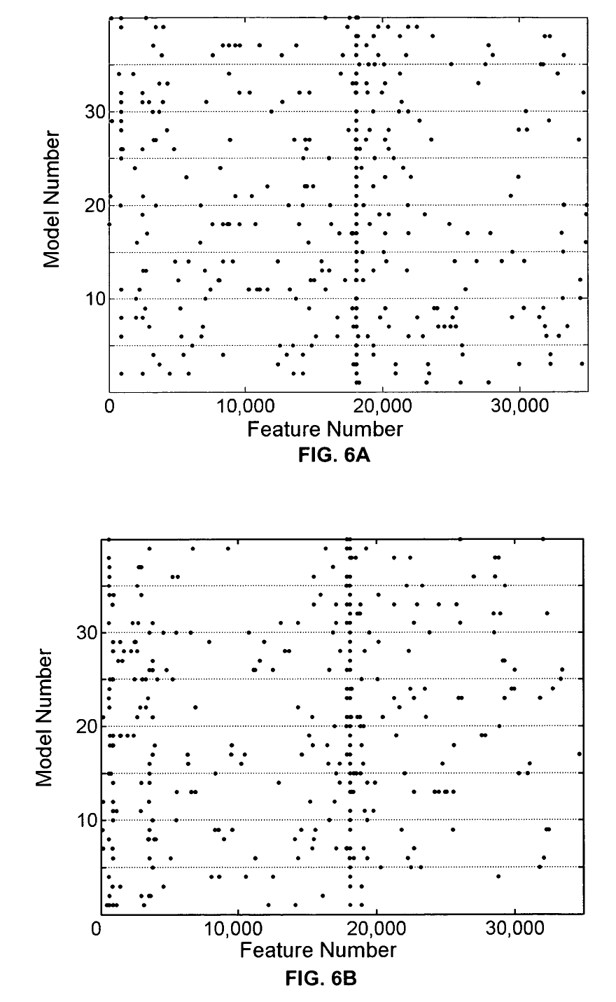

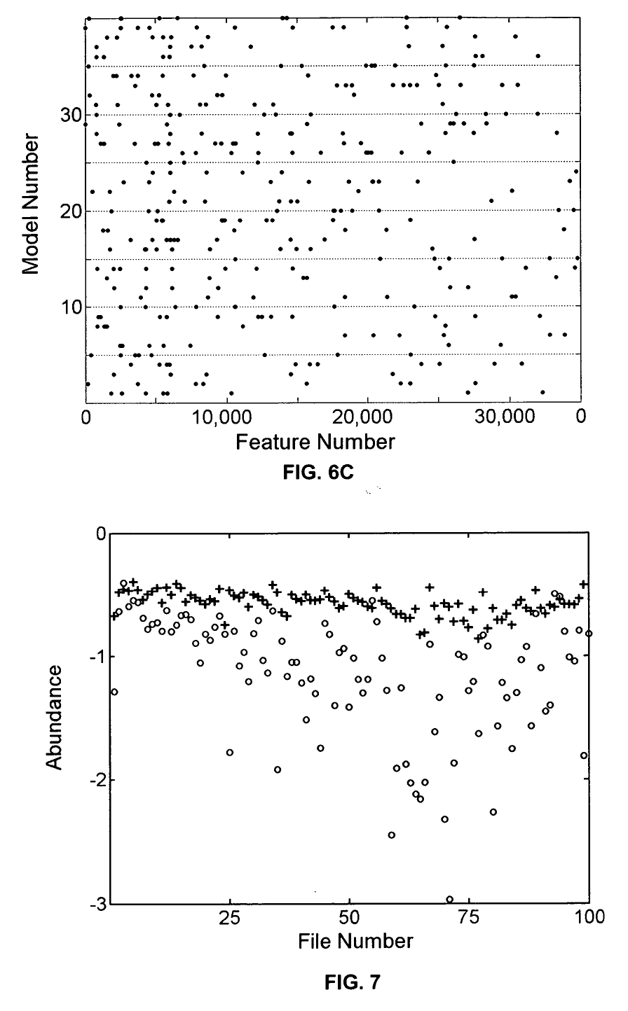

The biomarkers found across many models are displayed in FIGS. 6A-6C. FIG. 6A shows the biomarkers found in 40 models that allowed discrimination of B. subtilis and B. thuringiensis. Note that there is one biomarker that was selected in many of the models, which indicates that it is important in the discrimination of these two species. FIG. 6B shows a similar plot for B. subtilis and B. cereus, and again the same biomarker appears in many of these models as well. When comparing the models of B. cereus and B. thuringiensis (FIG. 6C), no biomarkers appear as frequently across all models, which is consistent with these two species being difficult to separate. To further examine one biomarker in particular that appears to be important in distinguishing B. subtilis from the other two species, the abundance value at that point in the raw data from B. subtilis and B. thuringiensis are graphed in FIG. 7. Even at the concentration of 5 k as shown, there is a clear trend of separation in the raw data. When the data are normalized to give the same total ion current for each spectrum, an identical plot is obtained.

To verify that B. cereus and B. thuringiensis tend to be harder to separate from each other than from B. subtilis due to their relatedness, several binary models were created that distinguish B. subtilis from a pool of B. cereus and B. thuringiensis files. Again the 5 k, 10 k and 80 k files for each species were combined and randomized prior to modeling. Models were created with a 50:100 training, 150:300 testing and 100:201 validation sets of spectra (B. subtilis: B. cereus and B. thuringiensis). The results for the six models yielding the highest accuracies are shown in Table 4, which compares validation results based on modeling B. subtilis against a combination of B. cereus and B. thuringiensis across 3 different spore concentrations (80 k spores, 10 k, and 5 k). 100 files of B. subtilis were modeled against 200 files of the other species (100 files of B. cereus and 100 files of B. thuringiensis). The data shown include number of features (F), match parameter (M), number of nodes (N), sensitivity (Sn), specificity (Sp), and accuracy (A). Sensitivity and specificity are calculated with respect to B. subtilis. The good classification obtained with these models shows that B. cereus and B. thuringiensis have biomarkers common to each other but that differ from B. subtilis.

TABLE 4
Comparison of validation results based on modeling B. subtilis against a
combination of B. cereus and B. thuringiensis
B. subtilis & (B. cereus and B. thuringiensis)

| Model | B | M | N | Sn | Sp | A |
|---|---|---|---|---|---|---|
| 1 | 10 | 0.9 | 39 | 95 | 86.9 | 92.3 |
| 2 | 11 | 0.9 | 32 | 95 | 85.9 | 92 |
| 3 | 12 | 0.9 | 35 | 93.5 | 83.8 | 90.3 |
| 4 | 8 | 0.9 | 23 | 92.5 | 84.8 | 90 |
| 5 | 7 | 0.9 | 27 | 93 | 81.8 | 89.3 |
| 6 | 11 | 0.8 | 8 | 90.5 | 84.8 | 88.7 |

As B. cereus and B. thuringiensis are the most difficult to classify, these two species were modeled at each concentration individually to determine if there is a concentration limit below which the species become indistinguishable. To generate these models, spectra were randomized and assigned into sets of 25:25 training, 50:50 testing, and 25:25 validation (B. cereus: B. thuringiensis). The models offering the highest accuracy were 60.8% at 5 k concentration, 64% at 10 k concentration, and 88% at 80 k concentration. Therefore, classification is more successful for these two closely related species when more spores are present.

Next, a set of 3-way comparisons were performed to classify all three groups from one another in a single model. For these models only the 80 k data were used, since it was determined that below that concentration B. cereus and B. thuringiensis are more difficult to distinguish. For each species the spectra were randomly assigned to a training set of 25, a testing set of 50, and a validation set of 25. The results are shown in Table 5. In Table 5a of 25 B. thuringiensis in the validation set, 2 were classified as B. cereus, 0 were classified as B. subtilis and 23 were correctly classified, an overall accuracy of 92%. Similarly the accuracy for B. subtilis was 88% and for B. cereus 52%. An overall accuracy of 77.3% was obtained. The overall accuracy of the second model is 73.3%, and the species accuracies are: B. subtilis 68%, B. thuringiensis 92%, and B. cereus 60%.

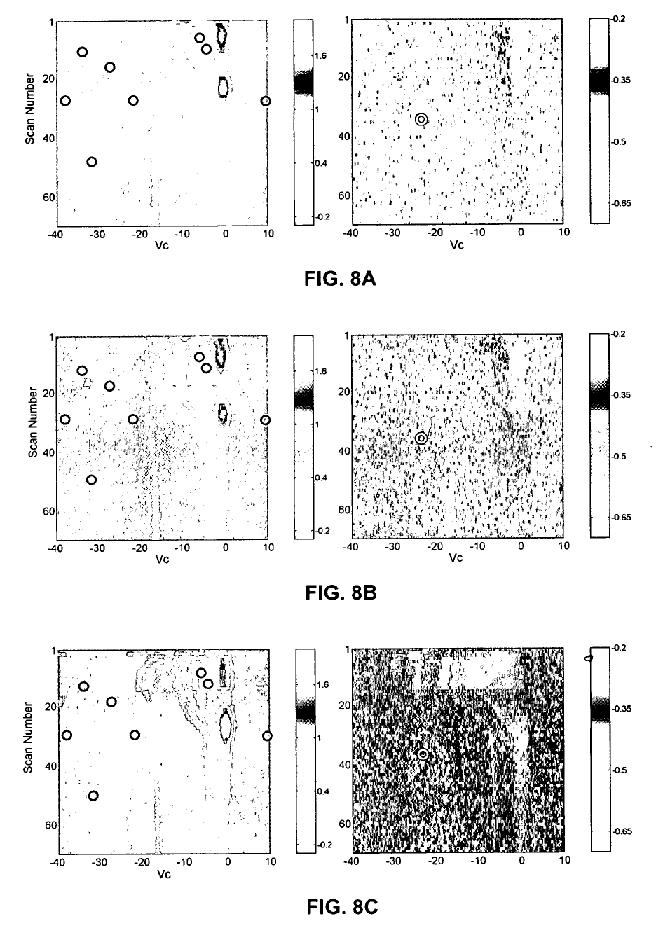

Representative spectra from the three species at 5,000 spore concentration are shown in FIGS. 8A-8C. The spectra are from 80,000 spores undergoing pyrolysis at 650° C. for 99.99 seconds. The positive ion spectrum is on the left, and the negative ion spectrum is on the right. The X-axis represents Vc (V), while the y-axis represents scan number. Features from the three-way model (a) are circled in black for positive spectra and in white for negative spectra. The raw data are shown here, but the biomarkers were selected based on their relative ratio after normalization between zero and one. The data from these experiments look very similar by eye, yet the pattern recognition algorithms were able to find biomarkers present in sufficient quantities to reliably distinguish the species from one another.

There is an increased interest in the development of portable, sensitive, real-time devices for the detection of biohazards. One particularly attractive development is the microDMx, a small device that detects ions which are separated by their mobility through an electric field. Its ability to specifically and sensitively detect various chemicals, including chemical weapons agents, has been demonstrated (92, 102-108). It has been shown that distinct microDMx spectra can be derived for three chemicals present in high concentrations in spores: dipicolinic acid, picolinic acid, and pyridine (109). The disclosed method has the ability to fractionate complex biological mixtures in a reliable and reproducible pattern that contains sufficient information to discriminate between closely related species of Bacillus spores. In particular, it has the ability to detect and distinguish B. subtilis, a spore-forming bacterium commonly found in environmental samples, from B. cereus and B. thuringiensis, which are closely related to B. anthracis, the causative agent of anthrax at a level below the reported median infectious dose. In particular, it has the ability to distinguish. B. subtilis from B. thuringiensis at an accuracy of 98.5%, B. subtilis from B. cereus at an accuracy of 92%, and B. thuringiensis and B. cereus at an accuracy of 69%. B. subtilis can also be distinguished from B. cereus and B. thuringiensis when the latter two are grouped together, indicating that there are biomarkers present in both B. cereus and B. thuringiensis that are the same, but different from the more distantly-related B. subtilis. The models were created across three concentrations so that biomarkers present across this entire range could be found. This ensures that the biomarkers will not dilute out at the lower concentrations, or that they will not saturate the detector at higher concentrations.

The samples were classified by analyzing the spectra generated by pyrolysis of live spores using ProteomeQuest, an algorithm that combines lead cluster mapping with a genetic algorithm to search for combinations of features in the spectra which, taken together, can discriminate between the different species. Each resulting feature combination represents a classification model. The six models with the highest accuracies for the binary comparisons are presented in Tables 3 and 5. Table 5 shows the results of modeling B. cereus versus B. subtilis versus B. thuringiensis in a single 3-way model. Twenty-five files of each species at the 80 k concentration were used for validation. Reading down the columns, one can determine how those 25 files were classified. Two models are shown. Model (a) using a Match of 0.9, contains 9 features and 22 Nodes, with an overall accuracy of 77.3% Model (b) using a match of 0.8, contains 12 features and 4 Nodes, with an overall accuracy of 73.3%.

TABLE 5
Results of modeling B. cereus versus B. subtilis versus
B. thuringiensis in a single 3-way model

| | Actual | | | |
|---|---|---|---|---|
| | | | | Total |
| Predicted | B. cereus | B. subtilis | B. thuringiensis | Classified |
| Model (a) | | | | |
| B. cereus | 13 | 3 | 2 | 18 |
| B. subtilis | 5 | 22 | 0 | 27 |
| B. thuringiensis | 7 | 0 | 23 | 30 |
| Total Actual Files | 25 | 25 | 25 | 75 |
| Accuracy | 52.0% | 88.0% | 92.0% | 77.3% |
| Model (b) | | | | |
| B. cereus | 15 | 8 | 2 | 25 |
| B. subtilis | 5 | 17 | 0 | 22 |
| B. thuringiensis | 5 | 0 | 23 | 28 |
| Total Actual Files | 25 | 25 | 25 | 75 |
| Accuracy | 60.0% | 68.0% | 92.0% | 73.3% |

The models are lead cluster maps defined in N-dimensional space, where N represents the number of features in a model. Each map consists of clusters, or nodes, which are unique to one species or another.

Classification of unknown samples is made by mapping the spectrum for the unknown into the existing map and determining the identity of the species by the node into which it falls. Different models differ in the number of features in the spectra, the number of nodes in the map, and the size of the decision boundary (Match) about the node. While many models of similar accuracy can be generated from the data, depending on the number of features and size of the match parameter (Tables 3 and 4), models with a high Match (0.9) and fewer nodes will be built from spectral features with the least variance within a species and may represent more robust models. However, the number of nodes can also reflect the number of discrete differences within the spectra of a species and models were developed with a high number of nodes that prove to be robust across many samples (data not shown). The decision as to which model is best to use becomes clearer as the models are challenged with more and more independent sets of spectra. Within the spectral datasets any features which are strong classifiers will be selected more frequently.

Within the Bacillus species examined there was one dominant classifier, feature 18097, that was selected by most of the 40 models created, and a number of less dominant ones selected by 5 or 6 of the models. The dominant feature appears many times in models distinguishing B. subtilis from one of the other two species. FIG. 7 is a plot of classifier feature 18097 p (Vc=−20.92, scan 3, within the negative ion region). The raw intensity of this feature was extracted from each of the 100 files for the 5 k concentration of B. subtilis (+) and B. thuringiensis (o) raw data. The data for each species shows a different distribution at this point. The classification algorithm finds data points such as this to aid in decision-making. While one feature alone can not completely discriminate the two species, the unique combination of features within a model does. Examining this feature across many files of B. subtilis and B. thuringiensis shows that indeed there is a trend of separation when plotting the raw abundance value at this point.

In addition to the binary comparisons, the disclosed method also has the ability to create a single model that can discriminate between 3 species (Table 5 and FIGS. 8A-8C). In this case, three-way modeling is generally less accurate than the 2-way modeling, in part because of the high genetic similarity of B. cereus and B. thuringiensis, seen in the binary modeling, which makes these spores very difficult to discriminate, especially when present in low quantities.

The disclosed methodology is widely applicable to similar situations. In addition to Bacillus spores, it may be applied to other spore formers that would be important to monitor, including B. cereus (a causative agent of food poisoning), Clostridium botulinum (botulism), C. perfingens (gas gangrene and food poisoning), C. tetani (tetanus), C. sordellii (diarrheal disease), and C. difficile (antibiotic-associated diarrhea and pseudomembranous colitis). The disclosed apparatus offers the potential for even further miniaturization. For example, a small pyrolysis oven may be mounted directly in-line with a microDMx device, to make the entire setup handheld. System control from an external computer can also be implemented readily, which would allow many of these units to be monitored from a single location. Finally, using other species it is possible to build a database of species-specific models. From the spectrum derived from a single environmental sampling a variety of biological agents might be identified against the database.

### REFERENCES

(All of the references, as well as other documents identified in the specification above, are hereby incorporated by reference in their entirety.)
(1) Gibson, T. D.; Prosser, O.; Hulbert, J. N.; Marshall, R. W.; Corcoran, P.; Lowery, P.; Ruck-Keene, E. A.; Heron, S. Sensors and Actuators B 1997, 44, 413-422.
(2) McEntegart, C. M.; Penrose, W. R.; Strathmann, S.; Stetter, J. R. Sensors and Actuators B 2000, 70, 170-176.
(3) Kharitonov, S. A.; Barnes, P. J. American Journal of Respiratory and Critical Care Medicine 2001, 163, 1693-1722.
(4) Phillips, M. Analytical Biochemistry 1997, 247, 272-278.
(5) Borland, C.; Cox, Y.; Higenbottam, T. Thorax 1993, 48, 1160-1162.
(6) Phillips, M.; Greenberg, J.; Cataneo, R. N. Free Rad. Res. 2000, 33, 57-63.
(7) Corradi, M.; Rubinstein, I.; Andreoli, R.; Manini, P.; Caglieri, A.; Poli, D.; Alinovi, R.; Mutti, A. American Journal of Respiratory and Critical Care Medicine 2003, 167, 1380-1386.
(8) McGrath, L. T.; Patrick, R.; Silke, B. European Journal of Heart Failure 2001, 3, 423-427.
(9) Sannolo, N. Journal of Chromatography, Biomedical Applications 1983, 276, 257-265.
(10) Phillips, M.; Gleeson, K.; Hughes, J. M. B.; Greenberg, J.; Cataneo, R. N.; Baker, L.; McVay, W. P. The Lancet 1999, 353, 1930-1933.
(11) Natale, C. D.; Macagnano, A.; Martinelli, E.; Paolesse, R.; D'Arcangelo, G.; Roscioni, C.; Finazzi-Agro, A.; D'Amico, A. Biosensors & Bioelectronics 2003, 00, 1-10.
(12) Carpagnano, G. E.; Barnes, P. J.; Geddes, D. M.; Hodson, M. E.; Kharitonov, S. A. American Journal of Respiratory and Critical Care Medicine 2003, 167, 1109-1112.
(13) Zechman, J. M.; Aldinger, S.; Labows, J. N. Journal of Chromatography, Biomedical Applications 1986, 377, 49-57.
(14) Labows, J. N.; McGinley, K. J.; Webster, G. F.; Leyden, J. J. Journal of Clinical Microbiology 1980, 12, 521-526.
(15) Chou, S.; Chedore, P.; Kasatiya, S. Journal of Clinical Microbiology 1998, 36, 577-579.
(16) Cundy, K. V.; Willard, K. E.; Valeri, L. J.; Shanholtzer, C. J.; Singh, J.; Peterson, L. R. Journal of Clinical Microbiology 1991, 29, 260-263.
(17) Miller, R. A.; Nazarov, E. G.; Eiceman, G. A.; King, A. T. Sensors and Actuators A 2001, 91, 307-318.
(18) Krylov, E.; Nazarov, E. G.; Miller, R. A.; Tadjikov, B.; Eiceman, G. A. Journal of Physical Chemistry A 2002, 106, 5437-5444.
(19) Miller, R. A.; Eiceman, G. A.; Nazarov, E. G.; King, A. T. Sensors and Actuators B 2000, 67, 300-306.
(20) Eiceman, G. A.; Krylov, E. V.; Krylova, N. S.; Nazarov, E. G.; Miller, R. A. Analytical Chemistry 2004, 76, 4937-4944.
(21) Snyder, A. P.; Dworzanski, J. P.; Tripathi, A.; Maswadeh, W. M.; Wick, C. H. Analytical Chemistry 2004, 76, 6492-6499.
(22) Schmidt, H.; Tadjimukhamedov, F.; Mohrenz, I. V.; Smith, G. B.; Eiceman, G. A. Analytical Chemistry 2004, 76, 5208-5217.
(23) Eiceman, G. A.; Karpas, Z. Ion Mobility Spectrometry; CRC Press: Boca Raton, 1994.
(24) Shvartsburg, A. A.; Tang, K.; Smith, R. D. Journal of American Society for Mass Spectrometry 2004, 15, 1487-1498.
(25) Wheatley, R. E. Antonie van Leeuwenhoek 2002, 81, 357-364.
(26) Petricoin III, E. F.; Ornstein, D. K.; Paweletz, C. P.; Ardekani, A. M.; Hackett, P. S.; Hitt, B. A.; Velassco, A.; Trucco, C.; Wiegand, L.; Wood, K.; Simone, C. B.; Levine, P. J.; Linehan, W. M.; Emmert-Buck, M. R.; Steinberg, S. M.; Kohn, E. C.; Liotta, L. A. Journal of the National Cancer Institute 2002, 94, 1576-1578.
(27) Orenstein, D. K.; Rayford, W.; Fusaro, V. A.; Conrads, T. P.; Ross, S. J.; Hitt, B. A.; Wiggins, W. W.; Veenstra, T. D.; Liotta, L. A.; Petricoin III, E. F. Journal of Urology 2004, 172, 1302-1305.
(28) Petricoin III, E. F.; Ardekani, A. M.; Hitt, B. A.; Levine, P. J.; Fusaro, V. A.; Steinberg, S. M.; Mills, G. B.; Simone, C.; Fishman, D. A.; Kohn, E. C.; Liotta, L. A. The Lancet 2002, 359, 572-577.
(29) Conrads, T. P.; Fusaro, V. A.; Ross, S.; Johann, D.; Rajapakse, V.; Hitt, B. A.; Steinberg, S. M.; Kohn, E. C.; Fishman, D. A.; Whiteley, G.; Barrett, J. C.; Liotta, L. A.; Petricoin III, E. F.; Veenstra, T. D. Endocrine-Related Cancer 2004, 11, 163-178.
(30) Krebs, M. D.; Mansfield, B.; Cohen, S. J.; Hitt, B. A.; Sonenshein, A. L.; Davis, C. E. Nature Methods Manuscript in Preparation.
(31) Krylova, N. S.; Krylov, E.; Eiceman, G. A.; Stone, J. A. Journal of Physical Chemistry A 2003, 107, 3648-3654.
(32) Holland Adaptation in Natural and Artificial Systems: an Introductory Analysis with Applications to Biology, Control, and Artificial Intelligence, Edn. 3.; MIT Press: Cambridge, Mass., 1992.
(33) Kohonen, T. Biol Cybern 1982, 43, 59-69.
(34) Stone, J. H. R., V. N.; Hoffman, G. S.; Specks, U.; Merkel, P. A.; Spiera, R.; Davis, J. C.; St. Clair, E. W.; McCune, J.; Ross, S.; Hitt, B. A.; Veenstra, T. D.; Conrads, T. P.; Liotta, L. A.; Petricoin, E. F. III. Arthritis and Rheumatism In Press.
(35) Elgaali, H.; Hamilton-Kemp, T. R.; Newman, M. C.; Collins, R. W.; Yu, K.; Archbold, D. D. Journal of Basic Microbiology 2002, 42, 373-380.
(36) Claeson, A.; Levin, J.; Blomquist, G.; Sunesson, A. Journal of Environmental Monitoring 2002, 4, 667-672.
(37) Zechman, J. M.; Labows, J. N. Canadian Journal of Microbiology 1985, 31, 232-237.
(38) Nelson, N.; Lagesson, V.; Nosratabadi, A. R.; Ludvigsson, J.; Tagesson, C. Pediatric Research 1998, 44, 363-367.
(39) Musa-Veloso, K.; Rarama, E.; F., C.; Curtis, R.; cunnane, S. Pediatric Research 2002, 52, 443-448.
(40) O'Neill, H. J.; Gordon, S. M.; Krotoszynski, B.; Kavin, H.; Szidon, J. P. Biomedical Chromatography 1987, 2, 66-70.
(41) Sharma, S. K.; Mohan, A. Indian Journal of Medical Research 2004, 120, 354-376.
(42) Madigan, M. T.; Martinko, J. M.; Parker, J. Biology of Microorganisms, 9th ed.; Prentice Hall: Upper Saddle River, N.J., 2000.
(43) Newcombe, R. G. Statistics in Medicine 1998, 17, 857-872.
(44) Feng, P. C. S.; Hartmann, P. A. Applied and Environmental Microbiology 1982, 43, 1320-1329.
(45) Hansen, W.; Yourassowsky, E. Journal of Clinical Microbiology 1984, 20, 1177-1179.
(46) Lambertus, G.; Elstro, A.; Sensenig, K.; Potkay, J.; Agah, M.; Scheuering, S.; Wise, K.; Dorman, F.; Sacks, R. Analytical Chemistry 2004, 76, 2629-2637.
(47) Ruzsanyi, V.; Baumbach, J. I.; Sielemann, S.; Litterst, P.; Westhoff, M.; Freitag, L. J. Chromatographia A Manuscript in press.
(48) Wilkins, K.; Larsen, K.; Simkus, M. Chemosphere 2000, 41, 437-446.
(49) Rose, L. J.; Simmons, R. B.; Crow, S. A.; Ahearn, D. G. Current Microbiology 2000, 41, 206-209.
(50) Korpi, A.; Pasanen, A.; Pasanen, P. Applied and Environmental Microbiology 1998, 2914-2919.
(51) Elliott-Martin, R. J.; Mottram, T. T.; Gardner, J. W.; Hobbs, P. J.; Bartlett, P. N. Journal of Agricultural Engineering Research 1997, 67, 267-275.
(52) Inglesby, T. V. et al. Anthrax as a Biological Weapon: Medical and Public Health Management. JAMA 271, 1735-1963 (1999).
(53) Friedlander, A. M. et al. Postexposure prophylaxis against experimental inhalation anthrax. J Infect Dis 167, 1239-1243 (1993).
(54) Smith, H. & Keppie, J. Observations on experimental anthrax; demonstration of a specific lethal factor produced in vivo by Bacillus anthracis. Nature 173, 869-870 (1954).
(55) Friedlander, A. M. in Textbook of Military Medicine: Medical Aspects of Chemical and Biological Warfare. (eds. R. Zajtchuk & R. F. Bellamy) 467-478 (Office of the Surgeon General, U.S. Department of the Army, Washington D.C.; 1997).
(56) Brookmeyer, R., Johnson, E. & Bollinger, R. Modeling the optimum duration of antibiotic prophylaxis in an anthrax outbreak. Proc Natl Acad Sci USA 100, 10129-10132 (2003).
(57) Phillips, A. P., Martin, K. L. & Broster, M. G. Differentiation between spores of Bacillus anthracis and Bacillus cereus by a quantitative immunofluorescence technique. J Clin Microbiol 17, 41-47 (1983).
(58) Phillips, A. P. & Martin, K. L. Quantitative immunofluorescence studies of the serology of Bacillus anthracis spores. Appl Environ Microbiol 46, 1430-1432 (1983).
(59) Phillips, A. P. & Martin, K. L. Investigation of spore surface antigens in the genus Bacillus by the use of polyclonal antibodies in immunofluorescence tests. J Appl Bacteriol 64, 47-55 (1988).
(60) Lampel, K. A., Dyer, D., Kornegay, L. & Orlandi, P. A. Detection of Bacillus Spores Using PCR and FTA Filters. J Food Protection 67, 1036-1038 (2004).
(61) Radnedge, L. et al. Genome Differences that Distinguish Bacillus anthracis from Bacillus cereus and Bacillus thuringiensis. Appl Environ Microbiol 69, 2755-2764 (2003).
(62) Helgason, E. et al. Bacillus anthracis, Bacillus cereus, and Bacillus thuringiensis—One Species on the Basis of Genetic Evidence. Appl Environ Microbiol 66, 2627-2630 (2000).
(63) Nakano, S. et al. A PCR Assay Based on a Sequence-Characterized Amplified Region Marker for Detection of Emetic Bacillus cereus. J Food Protection 67, 1694-1701 (2004).
(64) Iqbal, S. S. et al. A review of molecular recognition techniques for detection of biological threat agents. Biosens Bioelectron 15, 549-578 (2000).
(65) Patra, G., Sylvestre, P., Ramisse, V., Therasse, J. & Guesdon, J. L. Isolation of a specific chromosomic DNA sequence of Bacillus anthracis and its possible use in diagnosis. FEMS Immunol Med Microbiol 15, 223-231 (1996).
(66) Lee, M. A., Brightwell, G., Leslie, D., Bird, H. & Hamilton, A. Fluorescent detection techniques for real-time multiplex strand specific detection of Bacillus anthracis using rapid PCR. J Appl Microbiol 87, 218-223 (1999).
(67) Makino, S. I., Cheun, H. I., Watarai, M., Uchida, I. & Takeshi, K. Detection of anthrax spores from the air by real-time PCR. Lett Appl Microbiol 33, 237-240 (2001).
(68) Turnbull, P. C. et al. Bacillus anthracis but not always anthrax. JAppl Bacteriol 72, 21-28 (1992).
(69) Higgins, J. A., Ibrahim, M. S. & Knauert, F. K. Sensitive and rapid identification of biological threat agents. Ann NY Acad Sci 894, 130-148 (1999).
(70) Cooney, S. Rapid anthrax test development. Nature Medicine 7, 1265.
(71) Uhl, J. R. et al. Application of rapid-cycle real-time polymerase chain reaction for the detection of microbial pathogens: the Mayo-Roche Rapid Anthrax Test. Mayo Clin Proc 77, 673-680 (2002).
(72) Alam, S., Agarwal, G., Kamboj, D., Rai, G. & Singh, L. Detection of spores of Bacillus anthracis from environment using polymerase chain reaction. Indian J Exp Biol 41, 177-180 (2003).
(73) McBride, M. T. et al. Autonomous Detection of Aerosolized Bacillus anthracis and Yersinia pestis. Anal Chem 75, 5293-5299 (2003).
(74) Baeumner, A. J., Pretz, J. & Fang, S. A Universal Nucleic Acid Sequence Biosensor with Nanomolar Detection Limits. Anal Chem 76, 888-894 (2004).
(75) Ryu, C., Lee, K., Yoo, C., Seong, W. K. & Oh, H. B. Sensitive and Rapid Quantitative Detection of Anthrax Spores Isolated from Soil Samples by Real-Time PCR. Microbiol Immunol 47, 693-699 (2003).
(76) De, B. K. et al. A Two-Component Direct Fluorescent-Antibody Assay for Rapid Identification of Bacillus anthracis. Emerg Infect Dis 8, 1060-1065 (2002).
(77) Longchamp, P. & Leighton, T. Molecular recognition specificity of Bacillus anthracis spore antibodies. J Appl Microbiol 87, 246-249 (1999).
(78) Zhou, B., Wirsching, P. & Janda, K. D. Human antibodies against spores of the genus Bacillus: a model study for detection of and protection against anthrax and the bioterrorist threat. Proc Natl Acad Sci USA 99, 5241-5246 (2002).
(79) Quinlan, J. J. & Foegeding, P. M. Monoclonal Antibodies for Use in Detection of Bacillus and Clostridium Spores. Appl Environ Microbiol 63, 482-487 (1997).
(80) Hindson, B. J. et al. Development of and Automated Sample Preparation Module for Environmental Monitoring of Biowarfare Agents. Anal Chem 76, 3492-3497 (2004).
(81) Beverly, M. B., Basile, F. & Voorhees, K. J. A Rapid Approach for the Detection of Dipicolinic Acid in Bacterial Spores Using Pyrolysis/Mass Spectrometry. Rapid Commun Mass Spectrom 10, 455-458 (1996).
(82) Fox, A., Black, G. E., Fox, K. & Rostovtseva, S. Determination of carbohydrate profiles of Bacillus anthracis and Bacillus cereus including identification of O-methyl methylpentoses using gas chromatography-mass spectrometry. J Clin Microbiol 31, 887-894 (1993).
(83) Goodacre, R. et al. Detection of the Dipicolinic Acid Biomarker in Bacillus Spores Using Curie-Point Pyrolysis Mass Spectrometry and Fourier Transform Infrared Spectroscopy. Anal Chem 72, 119-127 (2000).
(84) Tripathi, A., Maswadeh, W. M. & Snyder, A. P. Optimization of quartz tube pyrolysis atmospheric pressure ionization mass spectrometry for the generation of bacterial biomarkers. Rapid Commun Mass Spectrom 15, 1672-1680 (2001).
(85) Smith, P. A. & MacDonald, S. Gas Chromatography using a resistively heated column with mass spectrometric detection for rapid analysis of pyridine released from Bacillus spores. J Chromatography A 1036, 249-253 (2004).
(86) Fergenson, D. P. et al. Reagentless Detection and Classification of Individual Bioaerosol Particles in Seconds. Anal Chem 76, 373-378 (2004).
(87) Cieslak, T. J. & Eitzen, E. M. Clinical and Epidemilogic Principles of Anthrax. Emerg Infect Dis 5, 552-555 (1999).
(88) Vasconcelos, D. et al. Pathology of inhalation anthrax in cynomolgus monkeys (Macaca fascicularis). Lab Invest 83, 1201-1209 (2003).
(89) Arakawa, E. T., Lavrik, N. V. & Datskos, P. G. Detection of anthrax simulants with microcalorimetric spectroscopy: Bacillus subtilis and Bacillus cereus spores. Applied Optics 42, 1757-1762 (2003).
(90) Schaeffer, P., Millet, J. & Aubert, J. P. Catabolic Repression of Bacterial Sporulation. Proc Natl Acad Sci USA 54, 704-711 (1965).
(91) Fouet, A. & Sonenshein, A. L. A target for carbon source-dependent negative regulation of the citB promoter of Bacillus subtilis. J. Bacteriol. 172, 835-844 (1990).
(92) Miller, R. A., Nazarov, E. G., Eiceman, G. A. & King, A. T. A MEMS radio-frequency ion mobility spectrometer for chemical vapor detection. Sensors and Actuators 91, 307-318 (2001).
(93) Krylova, N. S., Krylov, E., Eiceman, G. A. & Stone, J. A. Effect of Moisture on the Field Dependence of Mobility for Gas-Phase Ions of Organophosphorus Compounds at Atmospheric Pressure with Field Asymmetric Ion Mobility Spectrometry. J Phys Chem A 107, 3648-3654 (2003).
(94) Omstein, D. K. et al. Serum proteomic profiling can discriminate prostate cancer from benign prostates in men with total prostate specific antigen levels between 2.5 and 15.0 ng/ml. J Urol 172, 1302-1305 (2004).
(95) Conrads, T. P. et al. High-resolution serum proteomic features for ovarian cancer detection. Endocr Relat Cancer 11, 163-178 (2004).
(96) Stone, J. H. et al. A Serum Proteomic Approach To Gauging The State Of Remission In Wegener's Granulomatosis. Arthritis and Rheumatism In Press (2004).
(97) Petricoin, E. F. et al. Use of Proteomic Patterns in Serum to Identify Ovarian Cancer. The Lancet 359, 572-577 (2002).
(98) Petricoin, E. F. et al. Serum Proteomic Patterns for Detection of Prostate Cancer. J National Cancer Institute 94, 1576-1578 (2002).
(99) Adaptation in natural and artificial systems: an introductory analysis with applications to biology, control, and artificial intelligence, Edn. 3. (MIT Press, Cambridge (MA); 1992).
(100) Kohonen, T. Self-organized formation of topologically correct feature maps. Biol Cybern 43, 59-69 (1982).
(101) Krebs, M. D. et al. Detection of Biological and Chemical Agents using Differential Mobility Spectrometry (DMS) Technology. IEEE Sensors Journal In Review (2004).
(102) Miller, R. A., Eiceman, G. A., Nazarov, E. G. & King, A. T. A novel micromachined high-field asymmetric waveform-ion mobility spectrometer. Sensors and Actuators 67, 300-306 (2000).
(103) Miller, R. A., Eiceman, G. A., Nazarov, E. G. & King, A. T. in Solid-State Sensor and Actuator WorkshopHilton Head Island, SC; 2000).
(104) Eiceman, G. A. et al. Miniature radio-frequency mobility analyzer as a gas chromatographic detector for oxygen-containing volatile organic compounds, pheromones, and other inset attractants. J Chromatography 917, 205-217 (2001).
(105) Krylov, E., Nazarov, E. G., Miller, R. A., Tadjikov, B. & Eiceman, G. A. Field Dependence of Mobilities for Gas-Phase-Protonated Monomers and Proton-Bound Dimers of Ketones by Planar Field Asymmetric Waveform Ion Mobility Spectrometer (PFAIMS). J Phys Chem 106, 5437-5444 (2002).
(106) Schmidt, H., Tadjimukhamedov, F., Mohrenz, I. V., Smith, G. B. & Eiceman, G. A. Microfabricated differential mobility spectrometry with pyrolysis gas chromatography for chemical characterization of bacteria. Anal Chem 76, 5208-5217 (2004).
(107) Eiceman, G. A., Krylov, E. V., Krylova, N. S., Nazarov, E. G. & Miller, R. A. Separation of ions from explosives in differential mobility spectrometry by vapor-modified drift gas. Anal Chem 76, 4937-4944 (2004).
(108) Eiceman, G. A. et al. Differential Mobility Spectrometry of Chlorocarbons with a Micro-Fabricated Drift Tube. Analyst 129, 297-304 (2004).
(109) Davis, C. E. et al. in Transducers, Solid-State Sensors, Actuators and Microsystems, 12th Innational Conference on, Vol. 2 1233-12372003).

What is claimed is:

1. A method of identifying bacteria by analyzing a data stream that is obtained by processing a sample containing the bacteria, wherein the data stream has been abstracted to produce a sample vector that characterizes the data stream in a predetermined vector space containing at least one diagnostic cluster, the diagnostic cluster being associated with bacteria of known type, comprising:
determining whether the sample vector rests with the diagnostic cluster; and
if the sample rests within the diagnostic cluster, providing an indication that the bacteria are of the known type.

2. The method of claim 1, wherein the bacteria are selected from the group of genera consisting of Bacillus, Clostridium and Mycobacterium.

3. The method of claim 2, wherein the bacteria are selected from the group of species consisting of B. subtilis, B. cereus, B. thuringiensis, B. anthrasis), C. perfingens, C. tetani, C. sordellii, C. difficile, M smegmatis and M tuberculosis.

4. The method of claim 1, wherein the data stream is produced by a technique selected from the group consisting of mass spectrometry and high-field asymmetric waveform ion mobility spectrometry.

5. The method of claim 4, wherein the data stream is produced by a micromachined differential mobility spectrometer.

6. The method of claim 1, wherein the sample is selected from the group of sources consisting of headspace gas collected from a bacterial culture, bacterial culture fluid, and sputum, blood, urine, saliva, or breath collected from an animal or human patient.

7. A method of identifying bacterial spores by analyzing a data stream that is obtained by processing a sample containing the spores, wherein the data stream has been abstracted to produce a sample vector that characterizes the data stream in a predetermined vector space containing at least one diagnostic cluster, the diagnostic cluster being associated with spores of known type, comprising:
determining whether the sample vector rests with the diagnostic cluster; and
if the sample rests within the diagnostic cluster, providing an indication that the spores are of the known type.

8. The method of claim 7, wherein the sample containing spores is processed by pyrolosis.

9. The method of claim 7, wherein the spores are produced by bacteria selected from the group of genera consisting of Bacillus, Clostridium and Mycobacterium.

10. The method of claim 9, wherein the bacteria are selected from the group of species consisting of B. subtilis, B. cereus, B. thuringiensis, B. anthrasis, C. perfingens, C. tetani, C. sordellii, C. difficile, M. smegmatis and M tuberculosis.

11. The method of claim 7, wherein the data stream is produced by a technique selected from the group consisting of mass spectrometry and high-field asymmetric waveform ion mobility spectrometry.

12. The method of claim 11, wherein the data stream is produced by a micromachined differential mobility spectrometer.

13. A computer system containing a sample vector abstracted from a data stream that is obtained by processing a sample containing bacteria, the sample vector characterizing the data stream in a predetermined vector space containing at least one diagnostic cluster, the diagnostic cluster being associated with bacteria of known type.

14. A computer system containing a sample vector abstracted from a data stream that is obtained by processing a sample containing spores, the sample vector characterizing the data stream in a predetermined vector space containing at least one diagnostic cluster, the diagnostic cluster being associated with spores of known type.

15. A machine readable medium containing a sample vector abstracted from a data stream that is obtained by processing a sample containing bacteria, the sample vector characterizing the data stream in a predetermined vector space containing at least one diagnostic cluster, the diagnostic cluster being associated with bacteria of known type.

16. A machine readable medium containing a sample vector abstracted from a data stream that is obtained by processing a sample containing spores, the sample vector characterizing the data stream in a predetermined vector space containing at least one diagnostic cluster, the diagnostic cluster being associated with spores of known type.

17. A portable device for detecting one or more predetermined bacteria or bacterial spores in a sample, comprising:
a microfabricated differential mobility spectrometer configured to process the sample and output a data stream;
means for abstracting the data stream to produce a sample vector characterizing the data stream in a predetermined vector space containing at least one diagnostic cluster, the diagnostic cluster being associated with the predetermined bacteria or bacterial spores; and
means for determining whether the sample vector rests with the diagnostic cluster and, if the sample rests within the diagnostic cluster, for providing an indication that the sample contains the predetermined bacteria or bacterial spores.
